(19) United States
(12) Patent Application Publication
(10) Pub. No.: US 2022/0197008 A1
(43) Pub. Date: Jun. 23, 2022

(54) SYSTEM AND METHOD FOR TREATING ENDS OF OPTICAL FIBERS FOR USE IN AN ENDOSCOPE

(71) Applicant: Precision Optics Corporation, Inc., Gardner, MA (US)

(72) Inventors: Joseph N. Forkey, Princeton, MA (US); Bruce M. Radl, Stow, MA (US); David E. Chambers, Warren, MA (US)

(21) Appl. No.: 17/554,467

(22) Filed: Dec. 17, 2021

Related U.S. Application Data
(60) Provisional application No. 63/127,635, filed on Dec. 18, 2020.

(51) Int. Cl. G02B 23/24; G02B 6/04

(52) U.S. Cl. CPC G02B 23/2469; G02B 23/2423; G02B 23/2484; G02B 6/04

(57) ABSTRACT

Treated ends of optical fibers for use in an endoscope are disclosed. An end of an optical fiber, either raw cut or polished, is coated with an optical material that enables the end to be aligned without the need for conventional flush mounting techniques.

### RELATED APPLICATION

The present application claims the benefit of U.S. Provisional Patent Application Ser. No. 63/127,635, which was filed on Dec. 18, 2020, by Joseph N. Forkey et al. for SYSTEM AND METHOD FOR TREATING ENDS OF OPTICAL FIBERS FOR USE IN AN ENDOSCOPE, which is hereby incorporated by reference.

This application is related to U.S. Provisional Patent Application No. 63/043,189, entitled SYSTEM AND METHOD FOR TREATING THE END OF AN OPTICAL FIBER BUNDLE TO REDUCE LIGHT REFLECTION, filed on Jun. 24, 2020, the contents of which are hereby incorporated by reference.

This application is related to U.S. patent application Ser. No. 17/354,159, entitled SYSTEM AND METHOD FOR TREATING THE END OF AN OPTICAL FIBER BUNDLE TO REDUCE LIGHT REFLECTION, filed on Jun. 22, 2021, the contents of which are hereby incorporated by reference.

### BACKGROUND

### Technical Field

The present invention is directed to treating ends of optical fibers and more particularly to treating ends and loading of optical fibers for use in endoscopes.

### Background Information

Optical fibers are used to transmit light, or other electromagnetic radiation, along its length. To cause efficient coupling of the radiation into and out of an optical fiber or a bundle of optical fibers, both end faces are typically finished with a glossy surface that is achieved by optical polishing. The end faces are then typically mounted flush, i.e., in planar alignment with an aperture of a device. The smooth surface is also beneficial as it eliminates sharp edges that are present on the tip of a roughly finished optical fiber. These sharp edges may negatively impact the usefulness of the fiber due to the mechanical sharpness of the tips. The sharp edges also make the tips more prone to being damaged during use. Illustratively, a progression of finer and finer grit optical abrasives are used with a lapping tool to reduce the surface roughness of the end face until it achieves a suitably smooth and shiny surface that is substantially flat and free of pits and/or scratches. This polishing is illustratively performed in a multistep process that requires a substantial amount of time. The result is a surface on the end face that, similar to a polished lens, allows a substantial amount of light to be emitted with only Fresnel losses. The polished end is then aligned so that it is mounted flush with an aperture. This method is quite costly and requires a substantial amount of time to perform the plurality of rounds of polishing and precision to flush mount the optical fiber end.

The conventional polishing and flush mounting technique has worked well for reusable endoscopes and/or other devices where the additional costs required for processing the optical fiber end can be supported by the selling price of the reusable endoscopes. However, for single-use endoscopes (or other single use devices), where cost is critically important, these solutions are not practical. More generally, the conventional, multistep polishing and flush mounting technique may prevent the manufacture and/or assembly of low-cost devices where it is desirous to use optical fibers. Thus, there is a need for a low-cost and efficient alternative for the polished ends of optical fibers for use with low cost and/or single use devices.

### SUMMARY

The noted disadvantages of the prior art are overcome by providing novel treated ends of optical fibers for use in an endoscope. The treated ends of optical fibers in accordance with embodiments of the invention enabled improved emission of light. Further, the various embodiments described herein may be accomplished using substantially fewer resources than conventional optical fiber polishing and flush mounting, thereby reducing the overall cost of components that utilize optical fibers prepared in accordance with the various illustrative embodiments herein.

Illustratively, a bundle of one or more optical fibers has a proximal end that is aligned with an illumination source. The illumination source provides light rays that are captured by the proximal end and transmitted through the optical fibers. The light rays are then emitted from the distal ends of the optical fibers through illumination apertures at the distal end. Illustratively, the distal ends of the optical fibers may be raw cut and then coated with an optical material to improve the emission of light rays therefrom. The ends may be polished and then coated with an optical material to improve the transmission of light rays and to achieve alignment with an endpoint. Further, by varying the amount of material on the distal ends of the optical fibers, the fibers, though different lengths, may be emitting their light from a common surface, planar or otherwise.

### BRIEF DESCRIPTION OF THE DRAWINGS

The above and further advantages of the present invention are described herein in connection with the accompanying figures in which like reference numerals indicate identical or functionally similar elements, of which:

FIG. 1 is perspective view of an exemplary endoscope system in accordance with an illustrative embodiment of the present invention;

FIG. 2 is an enlarged view of a distal end of an endoscope in accordance with an illustrative embodiment of the present invention;

FIG. 3 is a perspective view of an end of an optical fiber in accordance with an illustrative embodiment of the present invention;

FIG. 4 is a cross-sectional view of an optical fiber in accordance with an illustrative embodiment of the present invention;

FIG. 5 is a cross-sectional view of an exemplary distal portion of an endoscope in accordance with an illustrative embodiment of the present invention;

FIG. 6A is a cross-sectional view of an end of an optical fiber bundle in accordance with an illustrative embodiment of the present invention;

FIG. 6B is a cross-sectional view of an end of an optical fiber bundle in accordance with an illustrative embodiment of the present invention;

FIG. 6C is a cross-sectional view of an end of an optical fiber bundle in accordance with an illustrative embodiment of the present invention;

FIG. 7 is a cross-sectional view of the end of an endoscope in accordance with an illustrative embodiment of the present invention; and

FIG. 8 is a flowchart detailing the steps of a procedure for finishing the ends of optical fibers for use in an endoscope in accordance with an illustrative embodiment of the present invention.

### DETAILED DESCRIPTION OF AN ILLUSTRATIVE EMBODIMENT

Endoscopes are well known in the art. Exemplary endoscopes are described in U.S. Pat. No. 6,139,490, entitled Stereoscopic Endoscope with Virtual Reality Viewing, U.S. Pat. No. 5,980,453, entitled Endoscope with Low Distortion, U.S. Pat. No. 7,758,498, entitled Endoscope with Relief of Axial Loading, and U.S. Patent Publication No. 2012/0212813 entitled Maximizing Illumination Fiber in an Endoscope, the contents of each of which are hereby incorporated by reference.

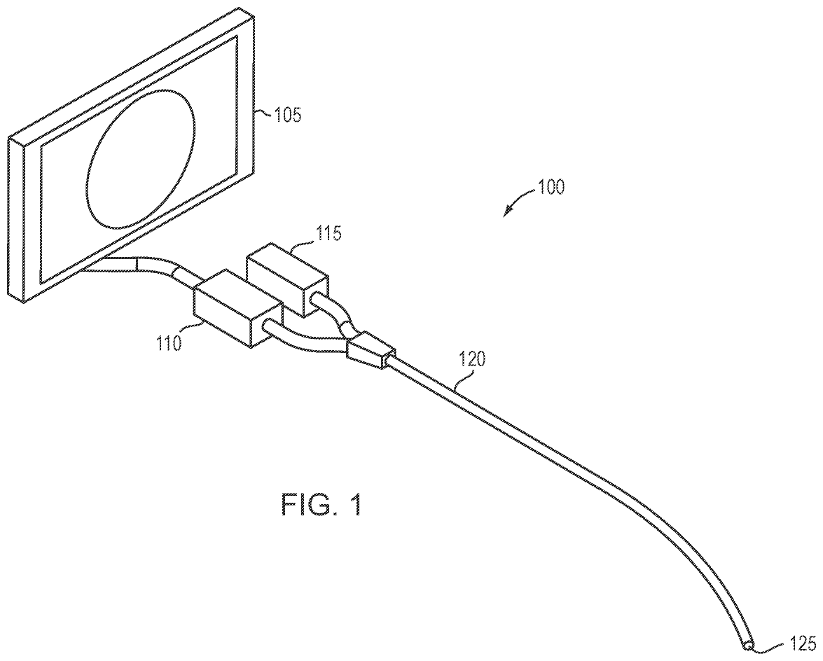

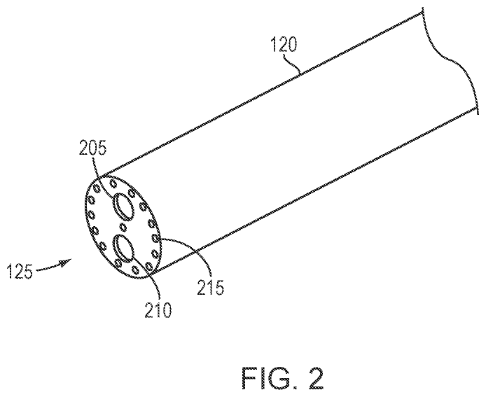

FIG. 1 is a general view of an exemplary endoscope system 100 in accordance with an illustrative embodiment of the present invention. A display 105 provides a view of the images received by endoscope. The display is powered by a vision processor 110 that illustratively receives images from the distal end 125 of the endoscope. An illumination source 115 provides illumination through the endoscope probe 120 to the distal end 125, typically through one or more optical fibers that may be organized into an optical fiber bundle. The distal end 125 is shown in greater detail in FIG. 2. It should be noted that while FIG. 1 references a flexible endoscope, the principles of the present invention may be utilized in other embodiments, e.g., rigid endoscopes. Therefore, the description of a flexible endoscope should be taken as exemplary only.

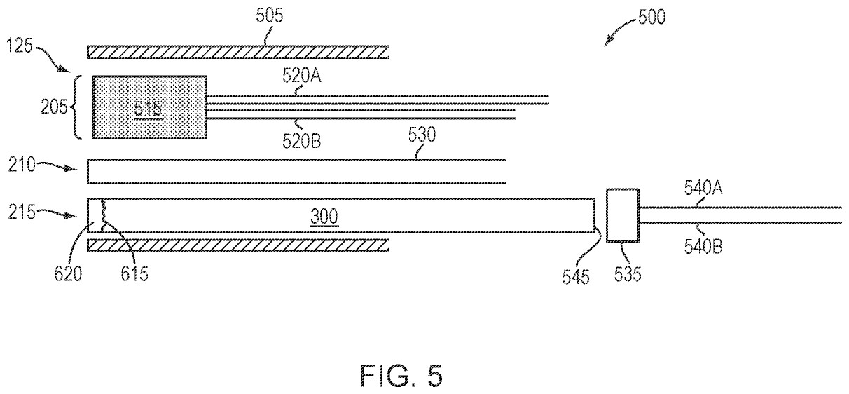

As seen in FIG. 2, the distal end 125 illustratively has two primary apertures 205 and 210. Illustratively, aperture 205 is used for an imaging device, such as a camera 515, described further below in reference to FIG. 5. Camera, or other imaging device, is used to obtain images to be fed back to display 105. In alternative embodiments, the aperture 205 may be used with a lens train to convey an image received at the distal end of the endoscope to an imaging device, or other sensors, located at the proximal end of the exemplary endoscope. Aperture 210 is utilized by a working channel for transmitting other surgical tools through the endoscope to perform medical procedures using the endoscope, as an irrigation channel, etc.

A plurality of illumination apertures 215 are provided on the distal end 125. The illumination apertures 215 are illustratively arranged in a predefined pattern. However, it should be noted that in accordance with alternative embodiments, the predefined pattern may vary from that shown and described herein in relation to FIG. 2. In accordance with other alternative embodiments, the illumination apertures 215 may be arranged in a random, non-predefined pattern. As such, the description of a specific pattern of apertures should be taken as exemplary only. Illustratively, each illumination aperture 215 is associated with an optical fiber or an optical fiber bundle, described further below, that is used to emit light to provide illumination of the area in front of the distal end of the endoscope. As will be appreciated by those skilled in the art, when an endoscope is inserted into a patient, images are obtained from imaging device 515 via aperture 205 while illumination is provided via illumination apertures 215.

It should be noted that while the exemplary endoscope is being described in relation to an imaging aperture, a working channel and a plurality of illumination apertures, the principles of the present invention may be implemented in endoscopes having differing configurations. Therefore, the description of specific components and arrangement thereof should be taken as exemplary only.

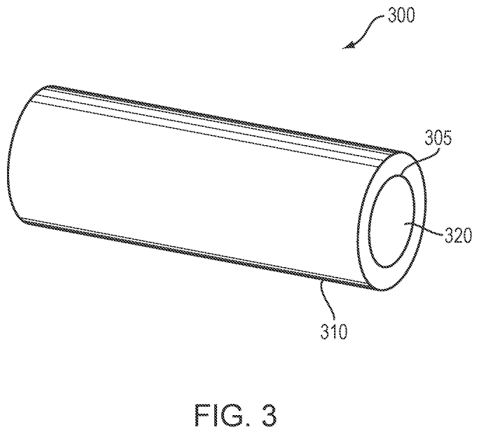

FIG. 3 is an exemplary perspective view of an optical fiber 300 that may be utilized in accordance with illustrative embodiments of the present invention. The optical fiber 300 illustratively comprises of an optical core 305 and a cladding 310 layer. It should be noted that in alternative embodiments of the present invention, additional layers may be utilized. In alternative embodiments, a plurality of optical fibers 300 may be arranged in a bundle with a further coating that encapsulates the entire bundle. Further, in alternative embodiments of the present invention the cladding 310 layer may be arranged in differing configurations, or may be absent altogether. As such, the arrangement of the exemplary optical fiber 300 shown in FIG. 3 should be taken as exemplary only.

Illustratively, the core 305 is made of glass or plastic and is clear so that light (or other electromagnetic radiation) will propagate through it. The core has an exemplary end surface 320 that may be used to capture or emit light, or other electromagnetic radiation, in accordance with illustrative embodiments of the present invention. As noted above, typically the end 320 is polished, often after it is loaded into the endoscope, using a multi-step polishing technique that takes a substantial amount of time and greatly increases the overall cost of finishing an optical fiber to achieve a polished surface aligned with the distal end of the endoscope. In accordance with illustrative embodiments of the present invention, a technique is described to treat the end 320 of the optical fiber core 305 to improve its optical collection or emission properties over those of a raw (i.e., not polished or treated) end and then to position it sub-flush to the end of the endoscope. More generally, the inventive concepts described herein may be utilized to improve performance of the optical fibers at the distal end of an exemplary endoscope while positioning them sub-flush to the distal end of such endoscope. It is expressly contemplated that the principles of the present invention may be utilized with either polished or non-polished fibers. Therefore, the description contained herein should be taken as exemplary only.

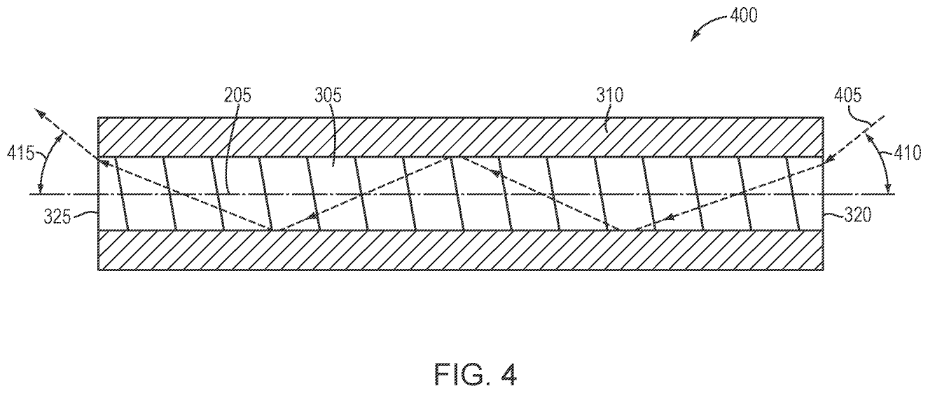

FIG. 4 is a cross-sectional view 400 of an exemplary optical fiber 400 in accordance with illustrative embodiment of the present invention. Exemplary light ray 405 enters the inner core 305 from a first end 320. As will be appreciated by those skilled in the art, there is a maximum light input angle 410 that can enter the core 305 and propagate via total internal reflection (TIR) through the core 305 based on the index of refraction properties of the core and cladding material. The cladding 310 protects the inner core material and prevents light from escaping. The light ray 405 propagates through the inner core material as indicated by the arrows and then exits the exemplary optical fiber at a second end 325. The maximum light output angle 415 that exits the core 305 is also characteristic of the index of refraction difference between the core and cladding materials.

As will be appreciated by those skilled in the art, not all light rays 405 that impact with end 320 are captured by the optical core 305. Some percentage of light rays are reflected off the end 320 and are not captured. Conventional polishing techniques for end 320 works to enable a very low percentage of light rays being reflected. Similarly, a conventionally polished end ensures that the number of light rays that are reflected back into the optical core when light is emitted remains low.

FIG. 5 is a schematic cross-sectional view 500 of an exemplary distal end 125 of an endoscope in accordance with an illustrative embodiment of the present invention. The distal end of the endoscope illustratively is formed within a metal tube 505 that provides rigidity for insertion into a patient's body. Aperture 205 is the entrance face of an imaging device 515, such as a camera, at the distal end 125 of the endoscope. The imaging device illustratively has cables 520 A,B that link the imaging device 515 with the vision processor. It should be noted that in alternative embodiments, a differing number of cables may be utilized to connect to the imaging device 515. In further alternative embodiments the cables may be substituted with rigid lenses or optical fibers to relay the image from a distal lens (gradient index for example) to a proximally located camera contained within the vision processor.

Aligned with aperture 210 is a working channel 530 that may be used for access by instruments, fluids, or energy. Illumination apertures 215 are aligned with optical fibers 300 that are then aligned, at their proximal end 545, with a light source 535. The light source may have one or more cables 540 A,B that feed back to the base of the endoscope in accordance with illustrative embodiments of the present invention.

It should be noted that while optical fibers 300 are shown as a singular unit, in illustrative embodiments, the optical fibers will be spaced around the other components at the distal end of the endo scope. Therefore, the depiction of a single optical fiber, or a bundle of optical fibers being in full alignment should be taken as exemplary only. As the optical fibers of the optical fiber bundle may extend from a single point at the proximal end to a plurality of differing points at the distal end, the optical fibers may not be aligned in a co-planar manner. As described further below, by treating the distal ends 615 of the optical fibers with varying amounts of material 620, the light from the distal ends of the optical fibers may be made to emit from a substantially co-planar surface in nominal alignment with the other elements of the distal end, e.g., lens cover and/or working channel 530. It should be noted that while end 615 of optical fiber 300 is shown as jagged, implying a raw cut end, the principles of the present invention may be utilized with either raw cut ends or polished ends. Therefore, the illustration of a raw cut end 615 should be taken as exemplary only.

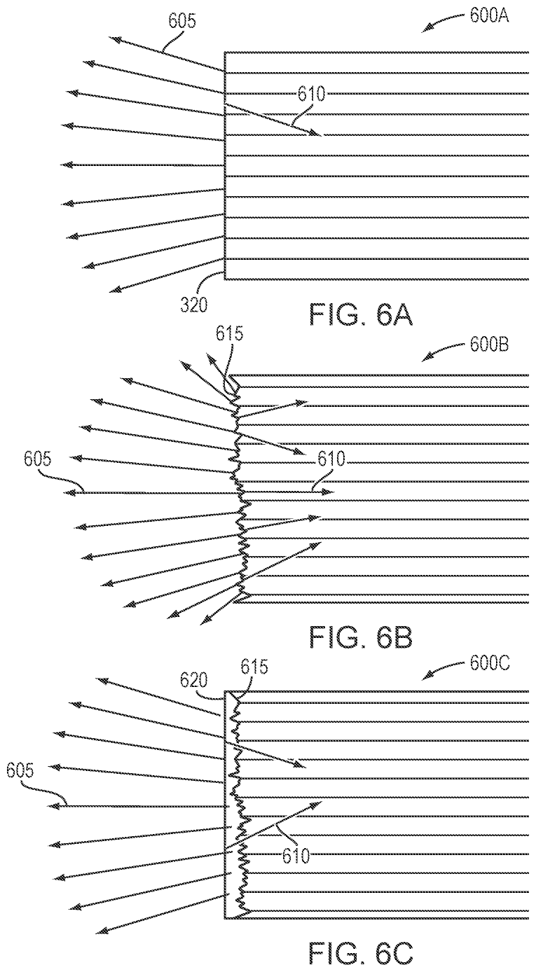

FIG. 6A is a side view 600A of an optical fiber bundle end illustrating light rays emitted from an optical fiber bundle with polished and flush optical fiber surfaces in accordance with an illustrative embodiment of the present invention. View 600A illustrates a substantial number of light rays 605 being emitted from polished end 320. Only a small number of light rays 610 are reflected back into the optical fiber bundle.

FIG. 6B is a side view 600B of an optical fiber bundle end illustrating light rays emitted from an optical fiber with a raw edge surface in accordance with an illustrative embodiment of the present invention. Light rays 605 are emitted but may be emitted at a variety of angles from raw end 615. A significant number of light rays 610 are reflected back into the bundle.

FIG. 6C is a side view 600C of an optical fiber bundle end illustrating light rays emitted from an end of an optical fiber bundle treated in accordance with an illustrative embodiment of the present invention. In exemplary view 600C, the distal ends have been coated with a material 620 in accordance with an illustrative embodiment of the present invention. While view 600C illustrates raw cut ends, the principles of the present invention may be utilized for polished ends that are not mounted flush with the end of the optical fiber bundle. Similar to FIG. 6A, a large number of light rays are emitted. The number of light rays 610 that are reflected back is larger than in FIG. 6A, but significantly less than in the case of a raw end in FIG. 6B.

In one embodiment of the present invention, the amount of light captured or emitted is increased as compared to the use of raw end, while avoiding the time and expense of multiple rounds of polishing as required by conventional techniques. Further, even when using polished fibers, the time and expense of mounting them flush may be reduced by mounting them sub-flush and utilizing the techniques of the present invention.

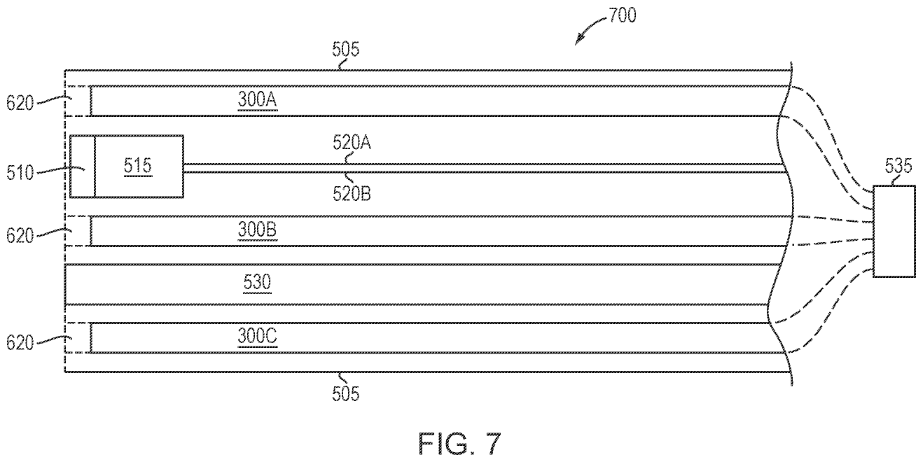

FIG. 7 is a cross-sectional view 700 of the end of an endoscope in accordance with an illustrative embodiment of the present invention. While view 500 shows a schematic view of the distal end of an exemplary endoscope, view 700 illustrates a more realistic view that incorporates the teachings of the present invention. In accordance with an illustrative embodiment of the present invention, a proximal end of a fiber optic bundle may be aligned with light source 535 to collect light rays for transmission to the distal end of the endoscope. Over the length of some portion of the endoscope, i.e., from light source 535 to exemplary apertures, the bundle is terminated into one or more smaller bundles or separate optical fibers that are then arranged on the distal end in a pattern to provide the desired illumination. The optical fibers may be rough finished on the distal ends with different lengths. Alternatively, the optical fibers may be polished as a tight bundle of fibers with equal lengths, but when positioned in the endoscope may extend along different paths from the proximal to distal end. In both of these cases the result is a set of optical fibers with distal end positions that are not co-planar. In addition, the distal ends of the optical fibers, whether co-planar or not, may be inside the distal end of the endoscope outside tube 505.

The material used to coat the distal ends of the optical fibers may vary in thickness so that the material coating the distal ends is substantially planar and co-planar with the end of the outside tube 505 which defines the distal end of the endoscope 700. By varying the thickness of the material on the distal ends of the optical fibers, a substantially flat optical emission surface results.

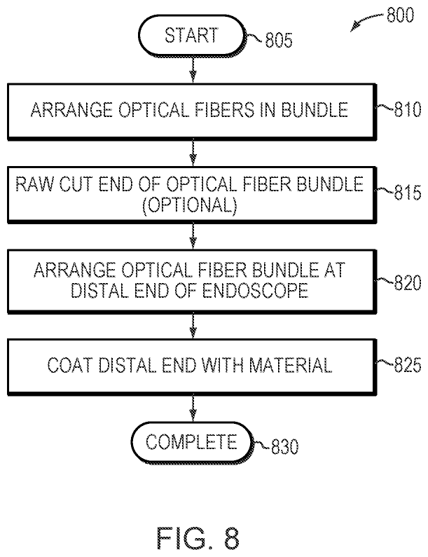

FIG. 8 is a flowchart detailing the steps of an exemplary procedure 800 for treating the end of an optical fiber bundle in accordance with an illustrative embodiment of the present invention. The procedure 800 begins in step 805 and continues to step 810 where the optical fibers are arranged in the bundle. Optical fibers may be organized into a bundle using a variety of known techniques. Illustratively, the optical fibers may be arranged during manufacturing, so the optical fibers share a common sheath. Alternatively, a plurality of optical fibers may be spatially arranged and then a coating potting material is applied to hold the optical fibers within the bundle. Alternatively, a plurality of optical fibers may be spatially arranged and then held in place manually, i.e., by hand. In alternative embodiments, an optical fiber bundle may comprise of a single optical fiber. In such alternative embodiments, a single optical fiber does not need further processing to be placed in a bundle. More generally, the arrangement of one or more optical fibers into an optical fiber bundle may include, e.g., arranging the optical fibers so that they are in a particular shaped pattern when viewing a cross-section through the diameter of the optical fiber bundle. Similarly, the cross-section of the optical fiber bundle may have a plurality of shapes.

Then, in optional step 815, one end of the optical fiber bundle is cut to generate a raw end surface. Illustratively, the raw end surface of the optical fiber bundle may be generated using any of a number of techniques, e.g., by cutting using a mechanical device, etc. The term cut should be construed broadly to include any method of terminating the end of the optical fiber bundle. Other than mechanical cutting, this may include, e.g., breaking, cleaving, laser cutting, chemical cutting, thermal cutting, etc. Subsequent to the initial cutting, further processing may also include grinding and polishing to any desired level of finish. Fibers may be processed individually or as a group in a bundle prior to arranging in a bundle where the ends are brought to a desired degree of alignment. This degree is variable and is allowed by the addition of the coating material.

In embodiments where the ends of the optical fiber bundle are polished, optional step 815 is not required. In such exemplary embodiments, the procedure may proceed from step 810 directly to step 820. In step 820, the optical fiber bundle is then arranged at the distal end of an exemplary endoscope. As noted above, the distal ends may be arranged in a predefined pattern at the distal end. Due to inexact method of cutting and the spatial arrangement of the optical fibers within the bundle, each individual optical fiber may have a slightly varying distance from the end of the endoscope.

In step 825, the end surface is coated with a material which extends from the distal end of the fiber to the distal end of the endoscope. Illustratively, the material is an epoxy, such as an optical adhesive used to bond or pot optical elements as is known to one skilled in the art. One example of such an optical adhesive is Norland Optical Adhesive 61. However, it should be noted that in accordance with illustrative embodiments of the present invention, the material may be a substance other than epoxy. Illustratively, any material that is transparent or translucent to the desired light wavelength range may be utilized. Therefore, the description of the use of an epoxy as the material to be utilized should be taken as exemplary only.

The procedure 800 then completes in step 830. Once procedure 800 has completed, the distal end of the optical fiber bundle has been coated. Additionally, other elements at the distal end may be coated with the same material to create a uniform surface on the distal end.

It should be noted that the various descriptions and embodiments described herein are exemplary only. While this description has been written in terms of certain materials, it should be noted that, in alternative embodiments, differing materials may be utilized. As such, the description of any specific materials should be taken as exemplary only. Further, while the description of the material being used to treat the ends of the optical fiber bundle is described as an epoxy, in alternative embodiments it is expressly contemplated that other materials may be utilized. Therefore, the description of the material being used as an epoxy should be taken as exemplary only.

What is claimed is:

1. An device comprising:
a fiber optic bundle, the fiber optic bundle having a set of optical fibers, wherein a distal end of a first optical fiber of the set of optical fibers is coated with a first transparent material; and
wherein the first transparent material extends from the distal end of the first optical fiber to a distal end of the endoscope.

2. The device of claim 1 wherein the distal end of the first optical fiber is a raw cut end.

2. The device of claim 1 wherein the coating of the first transparent material is not uniform in thickness for each of the optical fibers of the set of optical fibers.

3. The device of claim 1 further comprising an imaging device aligned with an aperture at the distal end of the endoscope.

4. The device of claim 1 wherein the coating of the first transparent material is terminated at a plane surface substantially coplanar with a distal face of the endoscope.

5. The device of claim 1 wherein a proximal end of the set of optical fibers is coated with a second transparent material.

6. The device of claim 5 wherein the first transparent material and the second transparent material are the same.

7. The device of claim 5 wherein the coating of the second transparent material is not uniform in thickness for each of the optical fibers of the set of optical fibers.

8. The device of claim 1 wherein the first transparent material comprises an epoxy.

9. The device of claim 1 wherein the coating of the first transparent material is shaped.

10. The device of claim 1 where the coating of the first transparent material is made to be glossy or polished in appearance.

11. The device of claim 1 where the coating of the first transparent material is made to be matte or diffuse in appearance.

12. The device of claim 1 where the coating of the first transparent material is made to be geometrically patterned to affect light distribution.

13. The device of claim 1 where the fibers terminate at a position so that they do not reach the plane of the exit face of the endoscope.

14. The endoscope of claim 13 where the raw illumination fibers have been processed to improve the surface finish by smoothing through grinding and polishing.

15. The endoscope of claim 13 where the coating allows light to transmit through it.

16. The endoscope of claim 13 where the coating is clear, without scattering properties.

17. The endoscope of claim 13 where the coating has scattering properties to alter a distribution of light.
